(19) United States
(12) Patent Application Publication
(10) Pub. No.: US 2017/0173348 A1
(43) Pub. Date: Jun. 22, 2017

(54) COPPER AND MAGNETIC THERAPY DEVICE

(71) Applicant: Julie A. Crews, Oklahoma City, OK (US)

(72) Inventor: Julie A. Crews, Oklahoma City, OK (US)

(21) Appl. No.: 14/973,435

(22) Filed: Dec. 17, 2015

(51) Int. Cl. A61N 2/06; A61D 9/00; A61D 99/00

(52) U.S. Cl. CPC A61N 2/06; A61D 99/00; A61D 9/00

(57) ABSTRACT

This application relates generally to a therapeutic device for providing a therapeutic effect to an animal, wherein the device has at least one magnet and a copper infused fabric with a contact side configured to contact a surface of the animal and an exterior side, and wherein the at least one magnet is positioned adjacent the exterior side of the copper infused fabric.

### FIELD OF THE INVENTION

The present disclosure relates generally to a device that implements copper and magnetic therapy for the treatment of animals.

### BACKGROUND

Magnetic therapy has been shown in studies to stimulate the circulation of blood, improve oxygen consumption of blood cells, and reduce inflammation and fluid retention, among other health benefits. These attributes are believed to promote healing and to reduce pain in targeted areas of the body where the magnetic therapy is applied.

Topical or dermal application of copper has also been shown to provide various health and therapeutic benefits. For example, copper may provide anti-fungal and antibacterial effects, as well as anti-inflammatory effects. The anti-fungal and antibacterial effects of copper can be beneficial in the healing of cuts, wounds, and hard-to-treat skin pathologies. The anti-inflammatory effects of copper can be beneficial in treating, for example, sore muscles, joints and arthritis.

The therapeutic benefits of applying both magnetic therapy and copper therapy simultaneously to a treatable area have not been provided. Thus, there is a need for a device that will target certain treatable areas of a body using the combinative therapeutic effects of both magnetic therapy and copper therapy. Such a device would provide a more dramatic and rapid response from the treated area than could be obtained from either form of therapy used consecutively or in isolation.

### BRIEF DESCRIPTION OF THE DRAWINGS

FIG. 1A is an exploded cross-sectional view of the therapeutic device showing a magnet, a spacer material, a copper infused fabric as a wrap and a surface of an animal.

FIG. 1B is a cross-sectional view of the magnet attached to the spacer material and the wrap being adjacent the surface of the animal.

FIG. 2A is an exploded cross-sectional view of the therapeutic device showing an overlay, a magnet, a spacer material, a copper infused fabric as a wrap and a surface of an animal.

FIG. 2B is a cross-sectional view of the overlay attached to the wrap securing the magnet in place over the spacer material and the wrap is adjacent the surface of the animal.

FIG. 3A is an exploded cross-sectional view of the therapeutic device showing a cover, a plurality of magnets, a copper infused fabric as a wrap and a surface of an animal.

FIG. 3B is a cross-sectional view of the cover attached to the wrap securing each magnet in place and the wrap is adjacent the surface of the animal.

FIG. 4A is an exploded cross-sectional view of the therapeutic device showing a plurality of overlays, a plurality of magnets, a copper infused fabric as a wrap and a surface of an animal.

FIG. 4B is a cross-sectional view of each overlay attached to the wrap, and each overlay secures a magnet in place and the wrap is adjacent the surface of the animal.

FIG. 5A is an exploded cross-sectional view of the therapeutic device showing a cover, a plurality of magnets, a plurality of copper infused fabric sections and a surface of an animal.

FIG. 5B is a cross-sectional view of each copper infused fabric section attached to the cover, and each copper infused fabric section secures a magnet in place and the copper infused fabric sections are adjacent the surface of the animal.

FIG. 6A is an exploded cross-sectional view of the therapeutic device showing a plurality of overlays, a cover, a plurality of magnets, a plurality of copper infused fabric sections and a surface of an animal.

FIG. 6B is a cross-sectional view of each overlay attached to each copper infused fabric section, and each overlay secures a magnet in place and the copper infused fabric sections are adjacent the surface of the animal.

FIG. 7 depicts an embodiment of a therapeutic device that covers the mid-section of a horse.

FIG. 8 depicts an embodiment of a therapeutic device that covers a portion of the front leg and knee of a horse.

FIG. 9 depicts an embodiment of a therapeutic device that covers a portion of the back leg and hock of a horse.

FIG. 10 depicts an embodiment of a therapeutic device that covers a portion of the hoof and heel of a horse.

### DETAILED DESCRIPTION

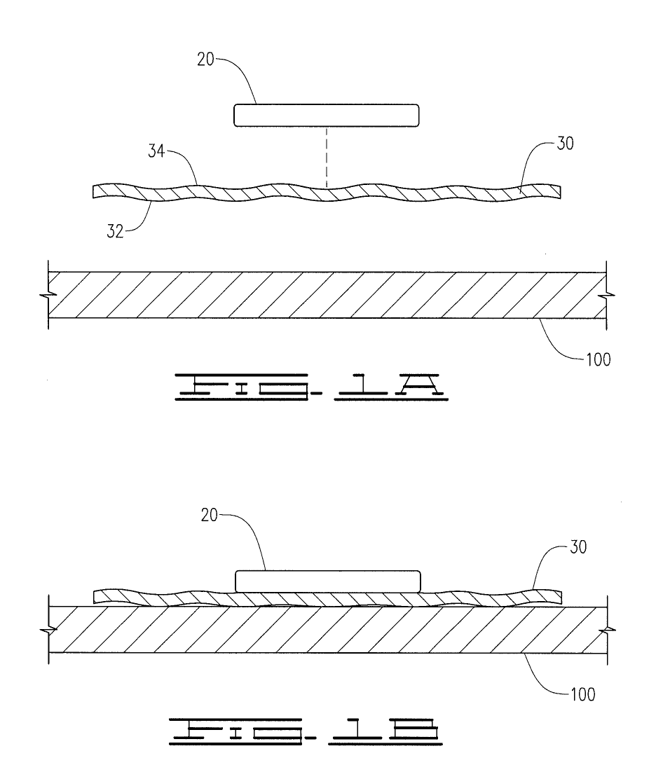

As shown in FIGS. 1A-10, the present disclosure is directed to a variety of embodiments of a therapeutic device that utilizes the benefits of magnetic therapy and copper therapy simultaneously in treating animals. Using both magnetic therapy and copper therapy together has shown dramatic increases in recovery time as compared to using either form of therapy consecutively or in isolation.

While each form of therapy provides its own set of health benefits, it has been shown that when used together, as described in the present disclosure, the combination enhances the individual therapeutic effects of each and reduces the response time to heal or otherwise recover. For example, magnetic therapy takes a certain amount of time to counteract stress, promote healing and reduce pain in targeted areas by, among other things, stimulating the circulation of blood, improving the oxygen consumption of blood cells, normalizing acid alkaline balances and reducing inflammation and fluid retention. Likewise, copper therapy takes a certain amount of time to promote healing of cuts, wounds, and hard-to-treat skin pathologies due to the anti-fungal and antibacterial effects of copper, as well as treat sore muscles, joints and arthritis due to the copper's anti-inflammatory effects. However, the effect of using both copper and magnetic therapy simultaneously, as disclosed herein, decreases an animal's response time to heal or otherwise recover by magnifying the individual therapeutic effects of each form of therapy.

The figures provided herein show some embodimets of the therapeutic device used with horses; however, the present disclosure is not limited to the treatment of horses and may include any animal that could benefit from the use of the therapeutic device disclosed herein.

As shown in FIG. 1A-10, the therapeutic device is depicted in a variety of embodiments. In each embodiment, the therapeutic device incorporates the use of at least one magnet 20 and a copper infused fabric 30.

In some embodiments, copper infused fabric 30 comprises a wrap 38 conformable to a shape of a portion of an animal being treated. The portion can include accupuncture points, meridians and major muscle groups. For example, in some embodiments, wrap 38 can conform to a portion of a leg, an area surrounding the hock or knee, a stifle, a hoof or paw or some other portion of an animal. Wrap 38 can also conform to a portion of a mid-section of an animal. In such an embodiment, wrap 38 can take the form of a blanket. The mid-section can include the shoulder, chest, withers, back, croup, dock, hip, flank and abdomen or some other portion of an animal, individually or in combination.

In other embodiments, copper infused fabric 30 comprises a plurality of strategically placed copper infused fabric sections 36. When used together, copper infused fabric sections 36 work collectively to target more than one portion of an animal. The portions can include accupuncture points, meridians and major muscle groups. For example, copper infused fabric sections 36 can target the shoulder, chest, withers, back, croup, dock, hip, flank and abdomen or some other portion of an animal.

Copper infused fabric 30 has a contact side 32 and an exterior side 34. Contact side 32 is positioned adjacent a surface 100 of an animal. In some embodiments, contact side 32 may actually contact surface 100 of an animal. Exterior side 34 of copper infused fabric 30 faces opposite surface 100 of an animal.

Copper infused fabric 30 is a fabric with copper bound to or infused in at least part of the fabric. Typically, the copper is bound to or infused in contact side 32 of copper infused fabric 30. In other embodiments, copper is bound to or infused in the entire fiber makeup of copper infused fabric 30.

Cutec Company provides a unique and proprietary method marketed under the federally registered trademark 29 Cu TEC® that utilizes the qualities of copper and permanently binds copper to the fiber matrix of a variety of fabrics. Cutec Company markets and sells these products to the public under designations such as CU 5000 or CU 2858, among others. The different designations relate to, among other things, different concentrations of copper that are infused in or bound to the fabric. The products marketed and sold by Cutec Company can be used in the therapeutic device of the present disclosure. However, copper infused fabric 30 is not limited to Cutec Company products, but can include other fabrics having copper bound to or infused therein.

Copper infused fabric 30 can be made from a variety of fabric types. For example, the fabric can be cotton, polyester, nylon, spandex or some other animal based, plant based, mineral based, synthetic based fabric or some combination. Copper infused fabric 30 can also include differing percentages of fabric infused with the copper. In some embodiments, from about 10% to about 100% of the total amount of thread used to create copper infused fabric 30 contains copper bound thereto or infused therein. In some embodiments, from about 20% to about 90% of the total amount of thread used to create copper infused fabric 30 contains copper bound thereto or infused thereon. In some embodiments, from about 30% to about 80% of the total amount of thread used to create copper infused fabric 30 contains copper bound thereto or infused thereon. In some embodiments, from about 40% to about 70% of the total amount of thread used to create copper infused fabric 30 contains copper bound thereto or infused thereon.

The therapeutic device of the present disclosure, as shown by the embodiments herein, incorporates the use of at least one magnet 20 that is positioned adjacent copper infused fabric 30. In other embodiments, more than one or a plurality of magnets 20 can be positioned adjacent copper infused fabric 30. Magnets 20 can be strategically positioned and spaced apart to target a variety of locations on an animal, including acupuncture locations, meridian locations or muscle groups. For example, in a horse the magnet(s) 20 can be positioned to target the poll, neck, shoulder, back, loin, withers, croup, dock, stifle, leg, pastern, heel, hoof or other designated area singly or in combination.

As shown in FIGS. 1A-6B, magnet 20 has a bottom side 22 and a top side 24. Bottom side 22 of magnet 20 is positioned adjacent exterior side 34 of copper infused fabric 30. In some embodiments, bottom side 22 of magnet 20 contacts exterior side 34 of copper infused fabric 30.

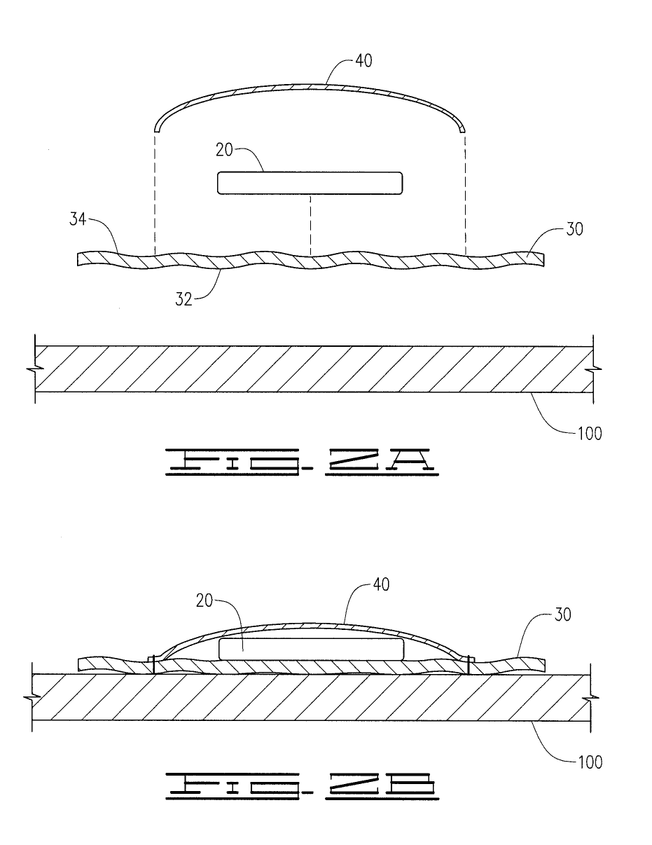

In other embodiments, as shown in FIGS. 1A-2B, a spacer material 60 can be positioned between magnet 20 and copper infused fabric 30. Spacer material 60 provides cushion between surface 100 and magnet 20 to prevent bottom edges 26 from rubbing on or placing unwanted pressure to surface 100 of an animal. Spacer material 60 can be made of an animal based, plant based, mineral based, synthetic based fabric or some combination. Although spacer material 60 is not included in every figure herein, spacer material 60 can be added to any one of the embodiments described herein or any other embodiment of the therapeutic device of the present disclosure.

In the therapeutic device of present disclosure, magnet 20 has a positive and a negative polarity. Magnet 20 may be used in a bipolar way or in a unipolar way. The term bipolar refers to a magnet that allows both poles—negative and positive—to face surface 100 of an animal at the same time. Unipolar refers to a magnet that is used in such a way that only one pole—either positive or negative (but not both)—faces surface 100 of an animal, while the opposite pole faces away from surface 100 of the animal. As shown in FIGS. 1A-10, magnet 20 is positioned such that the negative polarity of magnet 20 faces surface 100 of an animal. In this arrangment, bottom side 22 of magnet 20 faces surface 100 of the animal.

Magnet 20 can have any particular shape, including a circle, triangle, quadrilateral or any other conceivable shape. Magnet 20 is made from any ferromagnetic metal. Magnet 20 can also be made from more than one ferromagnetic metal. In some embodiments, magnet 20 is made of neodymium. In other embodiments, magnet 20 may be made of ceramic. It has been shown that neodymium magnets may absorb heat from sunrays, while ceramic magnets do not. Therefore, in some embodiments, neodymium magnets may be used on areas of an animal that are less prone to receive direct sunlight.

A magnet's strength is expressed in Gauss and is a measurement of the magnet's magnetic strength at a particular point near the magnet. In some embodiments, the magnitude of strength of magnet 20 can include those magnitudes above 2000 Gauss, above 3000 Gauss, above 4000 Gauss, above 5000 Gauss, above 6000 Gauss, above 7000 Gauss, above 8000 Gauss, above 9000 Gauss, above 10,000 Gauss, above 11,000 Gauss, above 12,000 Gauss, above 13,000 Gauss, above 14,000 Gauss, and above 15,000 Gauss. In other embodiments, the magnitude of strength of magnet 20 can include those magnitudes below 3000 Gauss, below 4000 Gauss, below 5000 Gauss, below 6000 Gauss, below 7000 Gauss, below 8000 Gauss, below 9000 Gauss, below 10,000 Gauss below 11,000, below 12,000 Gauss, below 13,000 Gauss, below 14,000 Gauss, and below 15,000 Gauss. In still other embodiments, magnet 20 can have a magnitude from between about 2000 Gauss to about 15,000 Gauss, about 5000 Gauss to about 15,000 Gauss, about 8000 Gauss to about 15,000 Gauss, or about 12,000 Gauss to about 15,000 Gauss.

FIGS. 1A-6B provide some embodiments of the therapeutic device of the present disclosure. However, FIGS. 1A-6B are not meant to limit the device to only these embodiments.

FIGS. 1A-1B depict an embodiment of the therapeutic device designated as numeral 10. In device 10 magnet 20 is positioned adjacent to wrap 38 which is comprised of copper infused fabric 30. In this embodiment, magnet 20 is not in contact with wrap 38. Instead, spacer material 60 is positioned between bottom side 22 of magnet 20 and exterior side 34 of wrap 38 to provide a cushion effect between magnet 20 and suface 100 of an animal.

As shown in FIG. 1B, contact side 32 of wrap 38 is positioned and configured to contact surface 100 of the animal. Bottom side 24 of magnet 20 attaches to spacer material 60 by any available known means, including by way of an adhesive, a fastener or Velcro®. Wrap 60 attaches to exterior side 34 of wrap 38 by any available known means, including by being sewn, heat-sealed, stapled, velcroed, glued, fastened or otherwise connected. In this embodiment, wrap 38 can extend in each direction to a predetermined size in order to conform to a portion of an animal. FIG. 1B depicts device 10 in a completed state. When in a completed state, device 10 can provide both magnetic and copper therapy simultaneously to surface 100 of an animal.

FIGS. 2A-2B depict an embodiment of the therapeutic device designated as numeral 11. Device 11 is depicted as having an overlay 40 positioned over magnet 20 with spacer material 60 positioned between magnet 20 and wrap 38.

Overlay 40 is defined as a piece of material that is placed over one magnet 20 or more than one closely placed magnets 20. In some embodiments, overlay 40 can be used to secure in place a magnet 20 or more than one closely placed magnets 20 adjacent copper infused fabric 30. In other embodiments, overlay 40 can be used to secure in place a magnet 20 or more than one closely placed magnets 20 to wrap 38. In still other embodiments, overlay 40 can be used to secure in place a magnet 20 or more than one closely placed magnets 20 to a copper infused fabric section 36. Overlay 40 can also be used to provide an added protective and/or decorative layer on the outside of the therapeutic device of the present disclosure. Overlay 40 can be made of any animal based, plant based, mineral based, synthetic based material or some combination thereof.

In FIG. 2A, magnet 20 is positioned between overlay 40 and spacer material 60. Spacer material 60 is positioned between bottom side 22 of magnet 20 and exterior side 34 of wrap 38 to provide a cushion effect between magnet 20 and suface 100 of an animal. Contact side 32 of wrap 38 is positioned and configured to contact surface 100 of the animal.

As shown in FIG. 2B, overlay 40 secures magnet 20 in place by attaching directly to wrap 38 with stitches 70. In other embodiments, overlay 40 can attach to wrap 38 by any available known means, including by being sewn, heat-sealed, stapled, velcroed, glued, fastened or otherwise connected. Magnet 20 can be attached to spacer material 60 by any available known means, including by way of an adhesive, a fastener or Velcro®. In other embodiments, magnet 20 is not attached to spacer material 60. Wrap 38 can extend in each direction to a predetermind size in order to conform to a portion of an animal. FIG. 2B depicts device 11 in a completed state which can provide both magnetic and copper therapy simultaneously to surface 100.

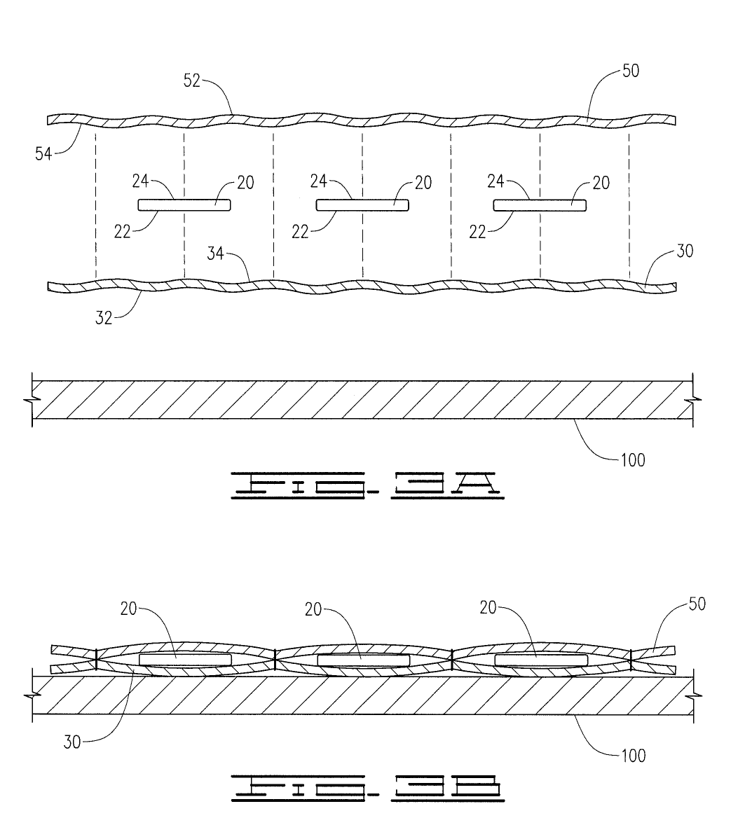

FIGS. 3A-3B depict an embodiment of the therapeutic device designated as numeral 12. Device 12 has a cover 50, a plurality of magnets 20 and wrap 38.

Cover 50 is a piece of material that provides structural support for and covers more than one spaced-apart magnet 20. Cover 50 is made of a flexible material conformable to a portion of an animal being treated. For example, cover 50 can be a mesh material or some other less porous material. Cover 50 can be made of an animal based, plant based, mineral based, synthetic based material or some combination thereof In some embodiments, copper can be bound to or infused in cover 50. As shown in FIG. 3A, cover 50 has an outer side 52 and an inner side 54.

In the embodiments of FIGS. 3A-3B, magnets 20 are positioned between cover 50 and wrap 38 such that top side 24 of magnet 20 is adjacent inner side 54 of cover 50 and bottom side 22 of magnet 20 is adjacent exterior side 34 of wrap 38. In this embodiment, the negative polarity of each magnet 20 faces the animal.

FIG. 3B depicts device 12 in a completed state which can provide both magnetic and copper therapy simultaneously to surface 100. Cover 50 and wrap 38 are attached with stitches 70. The stitching also secures magnets 20 in place. Although not shown, the stitching is placed around each magnet 20. Cover 50 can be attached to wrap 38 by any available known means, including by being sewn, heat-sealed, stapled, velcroed, glued, fastened or otherwise connected. As shown, device 12 is positioned such that contact side 32 contacts surface 100 of the animal.

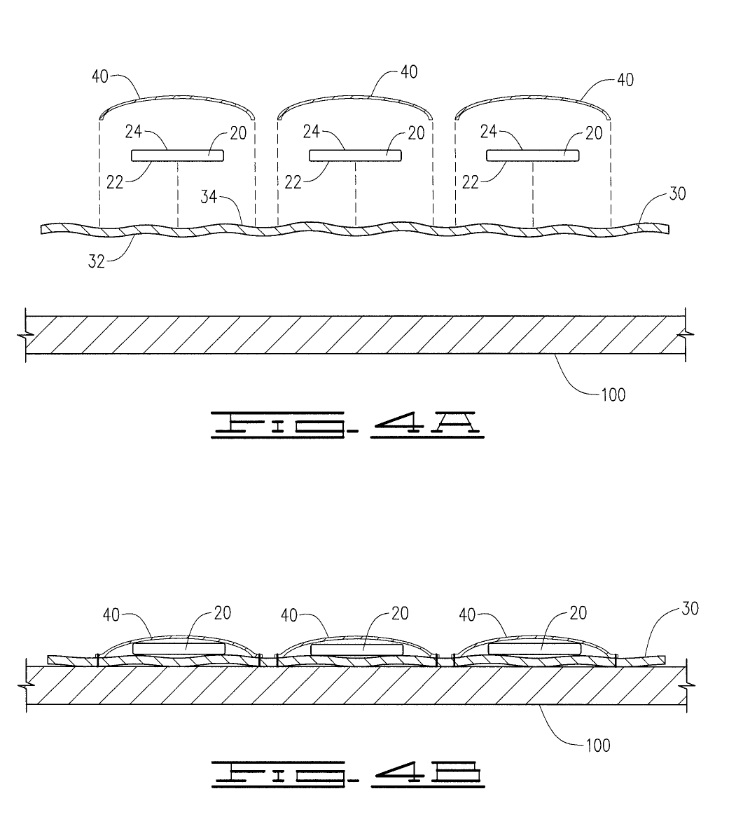

FIGS. 4A-4B depict an embodiment of the therapeutic device designated as numeral 13. Device 13 shows a plurality of overlays 40, a plurality of magnets 20 and wrap 38.

FIG. 4A depicts magnets 20 positioned between overlays 40 and wrap 38 such that top side 24 of each magnet 20 is adjacent a single overlay 40 and bottom side 22 of each magnet 20 is adjacent exterior side 34 of wrap 38. Each overlay 40 is positioned over a magnet 20 such that upon attaching each overlay 40 to wrap 38, each magnet 20 is secured in place. As shown, each overlay 40 is attached to wrap 38 with stitches 70. Stitches 70 are found around the outside perimeter of each overlay 40. In other embodiments, overlay 40 can be attached to wrap 38 by any available known means, including by being sewn, heat-sealed, stapled, velcroed, glued, fastened or otherwise connected. FIG. 4B depicts device 13 in a completed state which can provide both magnetic and copper therapy simultaneously to surface 100. FIG. 4B shows device 13 positioned and configured such that contact side 32 contacts surface 100 of the animal.

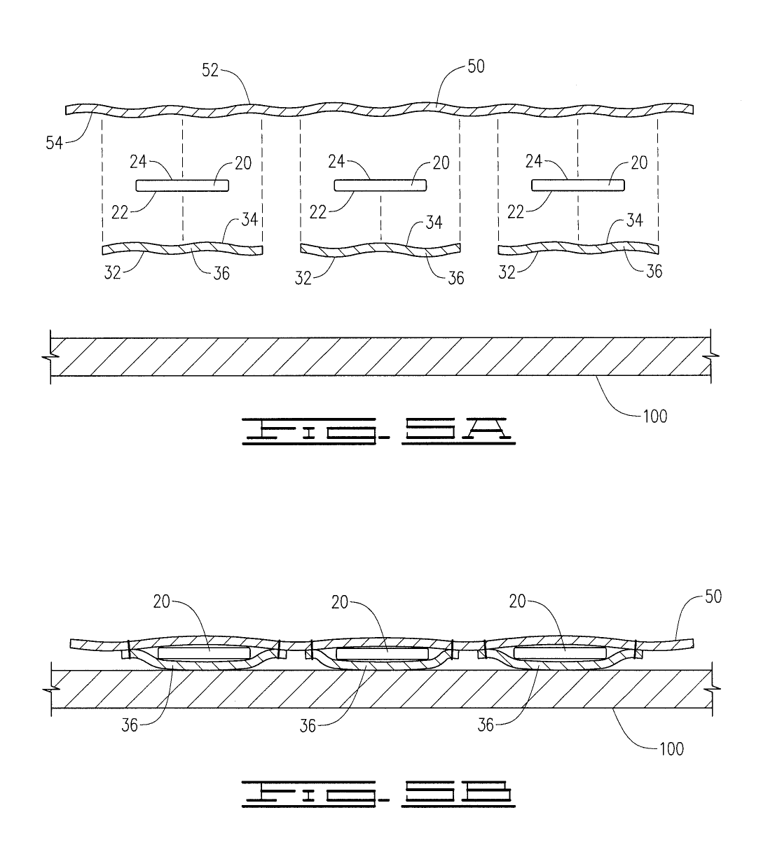

FIGS. 5A-5B depict an embodiment of the therapeutic device designated as numeral 14. Device 14 has a plurality of magnets 20, a plurality of copper infused fabric sections 36 and cover 50.

As shown in FIG. 5A, a magnet 20 is positioned between cover 50 and each copper infused fabric section 36. In this embodiment, top side 24 of magnet 20 is positioned adjacent inner side 54 of cover 50. Bottom side 22 of magnet 20 is positioned adjacent exterior side 34 of copper infused fabric section 36.

As shown in FIG. 5B, each copper infused fabric section 36 secures a magnet 20 in place by attaching to cover 50. FIG. 5B shows copper infused fabric sections 36 sewn to cover 50 with stitches 70. Stitches 70 occur around the outside perimeter of each copper infused fabric section 36. In other embodiments, copper infused fabric section 36 attaches to cover 50 by any available known means, including by being sewn, heat-sealed, stapled, velcroed, glued, fastened or otherwise connected. Device 14 is positioned and configured such that contact side 32 of each copper infused fabric section 36 contacts surface 100 of the animal. In a completed state, device 14 provides both magnetic and copper therapy simultaneously to surface 100 of the animal.

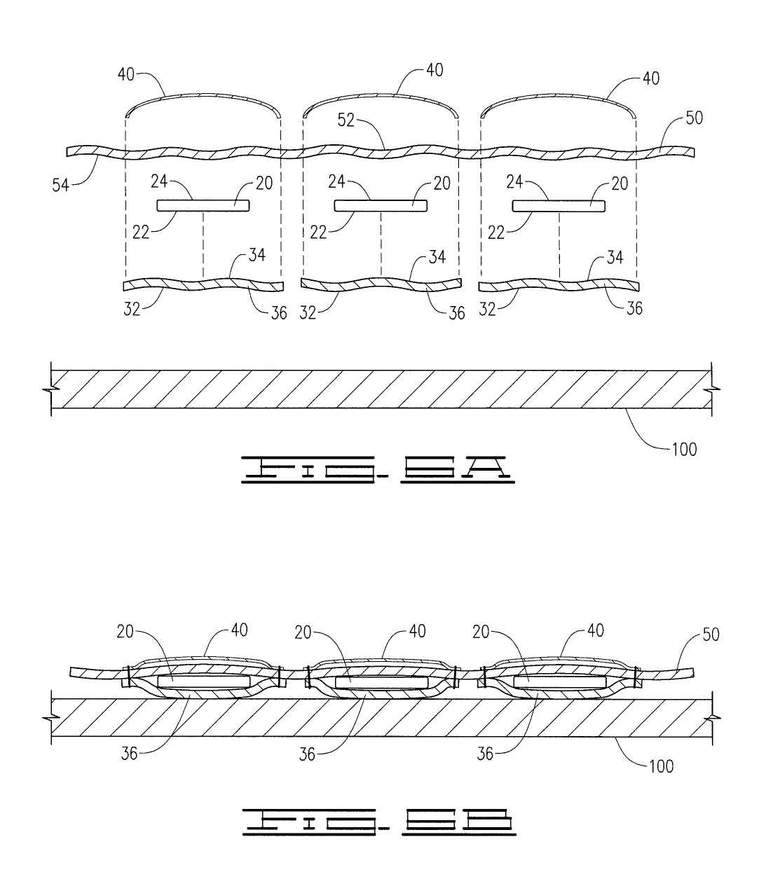

FIGS. 6A-6B depict an embodiment of the therapeutic device designated as numeral 15. Device 15 is depicted as having a plurality of copper infused fabric sections 36 and a plurality of magnets 20, a plurality of overlays 40 and cover 50.

As shown in FIG. 6A, cover 50 is positioned between each overlay 40 and each magnet 20. Each overlay is positioned over a magnet 20. Each magnet 20 is positioned between cover 50 and a copper infused fabric section 36. In other embodiments, cover 50 can be positioned between magnet 20 and copper infused fabric sections 36.

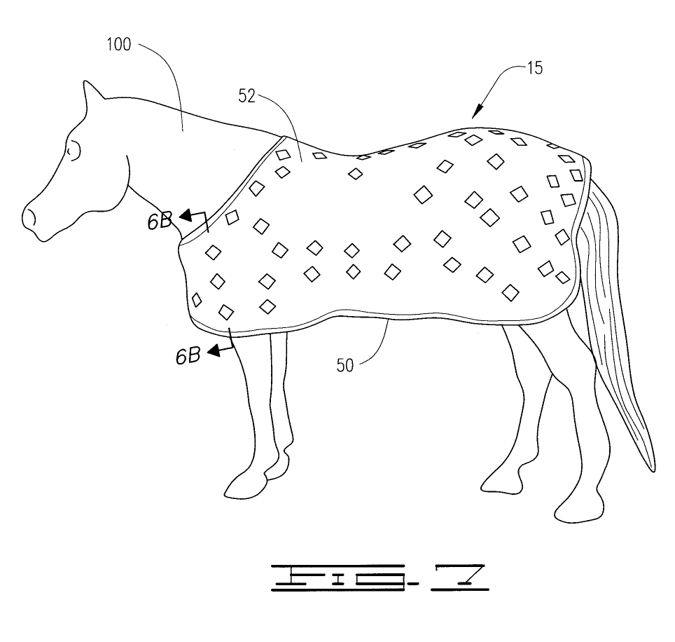

FIG. 6B is a cross-section of an embodiment of device 15 as shown in FIG. 7. Device 15 is not limited to targeting the mid-section of an animal as shown in FIG. 7, but can target any treatable area of an animal.

In FIG. 6B, device 15 has stitches 70 that extend from each overlay 40 to a copper infused fabric section 36. Stitches 70 occur around the perimeter of each magnet 20. In other embodiments, a fastener or other attachment can be used to secure overlays 40 to copper infused fabric sections 36. Device 15 is positioned and configured such that contact side 32 of each copper infused fabric section 36 contacts surface 100 of the animal. In a completed state, device 15 provides both magnetic and copper therapy simultaneously to surface 100.

FIGS. 7-10 show a variety of embodiments of the therapeutic device of the present disclosure. In each embodiment, the therapeutic device targets a different area of an animal. In these embodiments, the animal is a horse; however, as explained above, these embodiments are not meant to limit use of the therapeutic device to horses or to the areas targeted.

FIG. 7 shows device 15, as depicted in FIGS. 6A-6B, in the form of a blanket targeting multiple areas of an animal simultaneously. These areas include accupuncture points, meridians and major muscle groups. In FIG. 7, device 15 has a cover 50 that extends over a portion of the mid-section of a horse. A magnet 20 is positioned between inner side 54 of cover 50 and exterior side 34 of a copper infused fabric section 36. In this embodiment, the outer edges of an overlay 40 are sewn to the outer edges of a copper infused fabric section 36 to secure a magnet 20 in place. When in use, as shown, contact side 32 of each copper infused fabric section 36 contacts surface 100 of the animal. In this embodiment, cover 50 may be a mesh material or a less porous material that conforms to a portion of the horse. Copper can also be bound to or infused in cover 50.

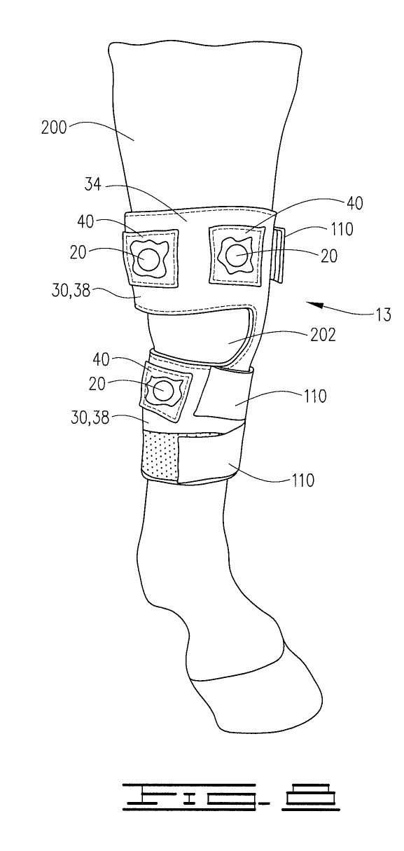

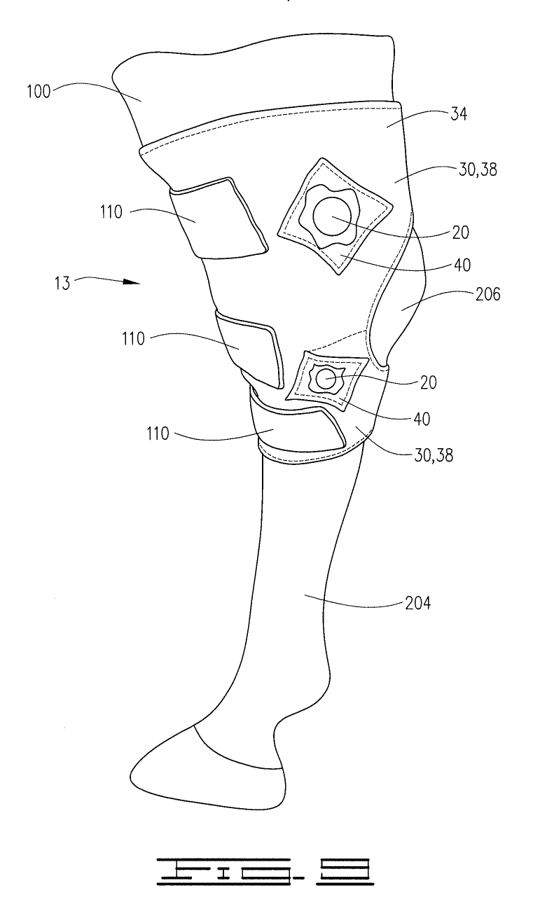

FIGS. 8-9 show device 13, as depicted in FIG. 4A-4B, in the form of a leg wrap. A magnet 20 is positioned between an overlay 40 and wrap 38 such that top side 24 of each magnet 20 is adjacent an overlay 40 and bottom side 22 of each magnet 20 is adjacent exterior side 34 of wrap 38. An overlay 40 is positioned over each magnet 20 so that upon attaching overlays 40 to wrap 38, each magnet 20 is secured in place. In this embodiment, the outer edges of each overlay 40 are sewn to wrap 38 to secure magnets 20 in place. When in use, as shown, contact side 32 of wrap 38 contacts surface 100 of the animal.

Device 13 of FIG. 8 is shown targeting a front leg 200 and knee 202 of the animal. Device 13 conforms around a portion of the front leg 200 and knee 202 and can be held in place with straps 110. Magnets 20 are spaced apart in such a way to specifically target the joints, muscles, ligaments and tendons that surround the front knee 200, or to target any other area to be treated.

Likewise, device 13, as depicted in FIG. 9, conforms around a portion of a back leg 204 and hock 206 of the animal and can be strapped in place using straps 110. Magnets 20 are also spaced apart in such a way to specifically target the joints, muscles, ligaments, and tendons that surround hock 206, or to target any other area to be treated.

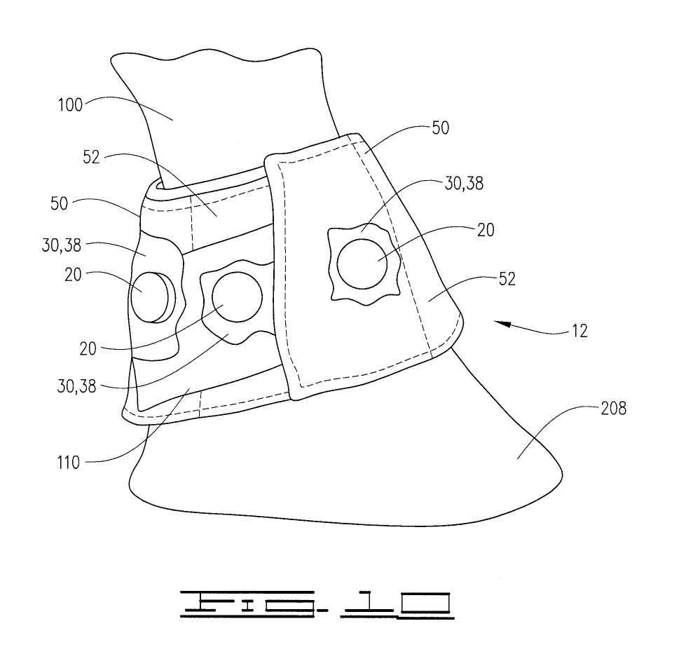

FIG. 10 shows device 12, as depicted in FIGS. 3A-3B, in the form of a hoof and heel wrap. Magnets 20 are positioned between cover 50 and wrap 38 such that top side 24 of each magnet 20 is adjacent inner side 54 of cover 50 and bottom side 22 of each magnet 20 is adjacent exterior side 34 of wrap 38. Cover 50 and wrap 38 are attached together in such a way as to secure each magnet 20 in place. In this embodiment, cover 50 and wrap 38 are sewn together. Device 12 conforms around a hoof 208 with contact side of wrap 38 facing the animal. Straps 110 strap to outer side 52 of cover 50 to secure device 12 in place. Magnets 20 are spaced apart in such a way to specifically target joints, muscles ligaments, and tendons around hoof 208 of the animal, or to target any other area to be treated.

Typically, response times to recover and heal are dependent on the type and severity of the injury. For example, torn tendons typically take longer to heal than strained tendons. In field trials, the therapeutic device of the present disclosure showed faster response times in both the initial stages and the latter stages of recovery and healing of injured areas when compared to response times using copper or magnetic therapy individually.

For example, a number of field trials conducted generally show the following response times for the initial stages and latter stages of reducing inflamation and relieving body and muscle soreness. When using the therapeutic device of the present disclosure to reduce inflamation and relieve body and muscle soreness, the response time to the initial stages of recovery generally occured around 30 minutes and the response time to the latter stages of recovery generally occured around 4 hours. When using magnetic therapy to reduce inflamation and relieve body and muscle soreness, the response time to the initial stages of recovery generally occured around 2 hours and the response time to the latter stages of recovery generally occured around 8-10 hours. When using copper therapy to reduce inflamation and relieve body and muscle soreness, the response time to the initial stages of recovery generally occured around 8-10 hours and the response time to the latter stages of recovery generally occur around 4-5 days. These results demonstrate that the therapeutic device of the present disclosure enhances individual therapeutic effects of each form of therapy that reduces the response time to recover in animals.

The present disclosure is well adapted to attain the ends and advantages mentioned, as well as those that are inherent therein. The particular embodiments disclosed above are illustrative only, as the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified, and all such variations are considered within the scope and spirit of the present disclosure. Also, the terms in the claims have their plain, ordinary meaning unless otherwise explicitly and clearly defined by the patentee.

What is claimed:

1. A device for providing a therapeutic effect to an animal comprising:
at least one magnet; and
a copper infused fabric with a contact side configured to contact a surface of the animal and an exterior side, wherein the at least one magnet is positioned adjacent the exterior side of the copper infused fabric.

2. The device of claim 1, wherein the at least one magnet has a negative polarity and the negative polarity faces the animal.

3. The device of claim 1, wherein the at least one magnet contacts the exterior side of the copper infused fabric.

4. The device of claim 1, further comprising an overlay positioned to secure the at least one magnet in place.

5. The device of claim 1, further comprising a spacer material positioned between the magnet and the copper infused fabric.

6. The device of claim 1, wherein the at least one magnet comprises a plurality of spaced-apart magnets and the plurality of spaced-apart magnets are positioned adjacent the exterior side of the copper infused fabric.

7. The device of claim 6, wherein the copper infused fabric comprises a plurality of copper infused fabric sections and at least one magnet of the plurality of spaced-apart magnets is positioned adjacent an exterior side of a single copper infused fabric section.

8. The device of claim 6, further comprising a cover that is conformable to a portion of the animal, wherein the plurality of spaced-apart magnets are positioned between the cover and the copper infused fabric.

9. The device of claim 6, further comprising a plurality of overlays, wherein each overlay is positioned to secure at least one magnet of the plurality of spaced-apart magnets in place.

10. A device for providing a therapeutic effect to an animal comprising:
a plurality of spaced-apart magnets, each magnet positioned adjacent an exterior side of a copper infused fabric, wherein the copper infused fabric has a contact side positioned adjacent the animal.

11. The device of claim 10, wherein the copper infused fabric comprises a wrap that is conformable to a portion of the animal.

12. The device of claim 11, wherein the portion of the animal is a leg.

13. The device of claim 11, further comprising a plurality of overlays, each overlay placed over at least one magnet of the plurality of spaced-apart magnets to secure the at least one magnet in place adjacent the exterior side of the wrap.

14. The device of claim 10, further comprising a cover that is conformable to a portion of the animal and the copper infused fabric comprises a plurality of copper infused fabric sections, and wherein at least one magnet of the plurality of spaced-apart magnets is secured between each copper infused fabric section and the cover.

15. The device of claim 14, further comprising a plurality of overlays attached to the cover, and wherein each overlay is placed over at least one magnet of the plurality of spaced-apart magnets.

16. The device of claim 14, wherein the cover is a copper infused fabric.

17. A device for providing therapeutic effects to an animal comprising:
a plurality of spaced-apart magnets;
a cover conformable to a portion of the animal; and
a plurality of copper infused fabric sections, wherein each copper infused fabric section has a contact side for contacting a surface of an animal and an exterior side and wherein each magnet is positioned adjacent the exterior side of each of the copper infused fabric sections and each copper infused fabric section is attached to the cover.

18. The device of claim 17, wherein the portion of the animal is a mid-section.

19. The device of claim 17, further comprising a plurality of spacer materials, wherein each spacer material is positioned between a magnet of the plurality of spaced-apart magnets and one of the copper infused fabric sections.

20. The device of claim 17, further comprising a plurality of overlays, wherein each overlay is attached to the cover and is positioned over at least one magnet of the plurality of spaced-apart magnets.
